(19) United States
(12) Patent Application Publication
(10) Pub. No.: US 2005/0164399 A1
(43) Pub. Date: Jul. 28, 2005

(54) Treating solution containing aluminium ion for staining protein and staining method using the same

(76) Inventors: Chulhun Kang, Jangan-gu (KR); Myungkoo Suh, Suwon-si (KR)

Correspondence Address: NIXON & VANDERHYE, PC, 901 NORTH GLEBE ROAD, 11TH FLOOR, ARLINGTON, VA, 22203, US

(21) Appl. No.: 10/506,182

(22) PCT Filed: Mar. 24, 2003
(86) PCT No.: PCT/KR03/00571

(30) Foreign Application Priority Data
Mar. 23, 2002 (KR) 10-2002-0015847

(51) Int. Cl. G01N 33/00

(52) U.S. Cl. 436/86; 204/450

(57) ABSTRACT

The present invention generally relates to treatment solutions for staining proteins including aluminum ions and staining methods using the same, and more specifically, to a treatment solution for staining proteins including aluminum ions, the treatment solution used when proteins are stained by a Commassie Brilliant Blue CBB stain, and a staining method using the same. The disclosed treatment solution including aluminum ions for CBB-staining proteins can decrease detecting limits, thereby improving the sensitivity and reducing staining time as well as omitting a de-staining process.

### TECHNICAL FIELD

The present invention relates to staining solutions for staining proteins comprising aluminium ions, and staining methods using the same.

### BACKGROUND OF THE INVENTION

Protein Staining important for analysis of experimental results in proteomics is a step of visualizing proteins in a two-dimensional space. Hereinafter, “dying” described in the specification refers to “staining”.

Two-dimensional gel electrophoresis (hereinafter, referred to as “2-D gel electrophoresis”) is a method of spreading out proteins in a cell by using a difference of molecular weight and electric charge, which are characteristics of proteins, in a two dimensional space. A comparative analysis of difference in expression of proteins under various conditions is based on the 2-D gel electrophoresis.

Conventionally, protein staining based on the 2-D gel electrophoresis in proteomics has been performed by staining techniques such as silver staining, fluorescence-based staining and CBB staining like G250 or R250.

The silver staining by the silver stain has a defect in that the sensitivity is dependent on types of proteins although showing excellent sensitivity.

However, the CBB staining is economically advantageous compared with other stains because they do not need an expensive instrument. In addition, the CBB staining has an easy experimental process and is relatively independent on types of proteins. As a result, the CBB staining has been effectively applied to analysis of proteins under various conditions by a 2-D gel electrophoresis.

Although the CBB staining is generally used in proteomics, it has a low detection limit of about 30 ng/band or more. A staining solution comprising ammonium ions is used to improve stain sensitivity by facilitating hydrophobic interaction between the stain and proteins in the CBB-staining. It takes about one to two days to stain and de-stain proteins, the method has a relatively long processing time.

### DETAILED DESCRIPTION OF THE INVENTION

There is provided a staining solution for staining proteins comprising aluminium ions, and a method for staining proteins using the same.

Hereinafter, the present invention will be described in detail.

There is provided a staining solution comprising aluminium ions when proteins are stained by a CBB-staining.

The aluminium ions (Al³⁺) having 3+ cations increase staining speed and strengthen interaction of the staining dye, CBB, with proteins.

The aluminium ion is preferably provided by aluminium salt selected from the group consisting of aluminium sulfate, aluminium chloride and aluminium acetate, most preferably aluminium sulfate.

It is preferably that the aluminium ion is present in an amount ranging from 1 to 40% of the total staining solution. If the amount of aluminium salt is less than 1%, effects on staining time and staining power decrease. However, if more than 40%, proteins are not identified because background part is thickly stained.

The staining solution comprises an alcohol compound in an amount ranging from 5 to 40% of the total staining solution. The alcohol compound is an essential element in staining solution for properly staining proteins. If the amount of alcohol compound is less than 5%, the staining is delayed. However, if more than 40%, the staining is not properly performed.

The alcohol compound is preferably selected from the group consisting of methanol, ethanol, propanol, iso-propanol, butanol and iso-butanol, more preferably ethanol.

Next, there is provided a method for staining proteins using the above staining solution comprising the aluminium salt, comprising the steps of:
(a) washing a gel with deionized water, the gel obtained by performing a certain degree of purified proteins with a Sodium Dodecyl Sulfate-Polyacrylamide Gel Electrophoresis (hereinafter, referred to as “SDS-PAGE”);
(b) treating the resulting gel with a primary solution and a secondary solution;
(c) treating the resulting gel in a tertiary solution comprising aluminium ions; and
(d) adding a quaternary solution comprising a CBB stain to the resulting third solution including the gel.

In the step (a), after subjecting a certain degree of purified proteins by SDS-PAGE, a gel removed from a glass plate is simply washed with de-ionized water.

In the step (b), the gel obtained from the step (a) is three times treated with a primary aqueous solution comprising 30% ethanol and 2% phosphoric acid, and then three times treated with a secondary aqueous solution comprising 2% phosphate.

In the step (c), the gel obtained from the step (b) is soaked in a tertiary aqueous solution comprising 1 to 40% aluminium ions, 5 to 40% alcohol compound and 2% phosphoric acid.

In the step (d), a quaternary aqueous solution comprising 2% G250 as a CBB stain solution and 0.2 g/L sodium azide is added to the tertiary solution where the gel obtained from the step (c) is soaked. Here, the quaternary solution is regulated to have the final concentration ranging from 0.5 to 2 When the size of gel is 7×10 cm, the amount of the primary through the quaternary solutions used is 50 ml, and when the size is 20×24 cm, the amount is 250 ml. The treatment time in the primary through the tertiary solutions is 30 minutes, respectively. The treatment time in the quaternary solution comprising the CBB stain ranges from 30 min. to 48 hours. A longer treatment time is applicable.

FIGS. 1a through 1d are figures illustrating the staining results of various proteins after subjecting the proteins isolated from a rabbit, E. coli, a bovine or an egg by two-dimensional gel electrophoresis to represent effects of the present invention.

FIG. 1a is a view illustrating the CBB-staining results of various proteins using a treatment solution comprising 15% aluminium ions according to the present invention, and FIG. 1b is a view illustrating the CBB-staining results of various proteins using a treatment solution comprising 5% aluminium ions according to the present invention.

On the other hand, FIG. 1c is a view illustrating the CBB-staining results of various proteins using a treatment solution comprising ammonium ions, and FIG. 1d is a view illustrating the acidic silver-staining results of various proteins using an acidic silver.

Each lane shown in FIGS. 1a through 1d represents molecular weight (unit: kDa) of each protein. The lanes sequentially represent rabbit skeletal muscle myosin (200 kDa), E. coli, phosphorylase b (116 kDa), rabbit skeletal muscle galactosidase (97 kDa), bovine serum albumin (66 kDa), egg white ovalbumin (45 kDa), and bovine carbonic anhydrase (31 kDa). Numbers on the upper side of the drawings represent a dilution degree of protein (serial 2-fold dilution), thus the number increases, the dilution degree increases.

When FIGS. 1a and 1b illustrating the CBB-staining results using a treatment solution comprising aluminium ions are compared with FIG. 1c illustrating the CBB-staining using a treatment solution comprising ammonium ions, it is shown that proteins of FIGS. 1a and 1b are well stained in case of high dilution fold. Here, it is shown that the present invention has greater sensitivity.

The detection limit of protein according to the present invention is less than 0.5 ng/band. A staining sensitivity is about 2 to 10 times improved when the treatment solution comprising aluminium ions is used than when the treatment solution comprising ammonium ions is used.

When FIGS. 1a and 1b illustrating the CBB-staining results using a staining solution comprising aluminium ions are compared with FIG. 1d illustrating the silver-staining results, the silver-staining is shown to depend on types of proteins because myosin proteins of the first lane are rarely stained while the CBB-staining is shown not to depend on types of proteins because all proteins are detected.

FIG. 2 is a graph illustrating relative variation in staining power dependent on treating time. When proteins are CBB-stained with the staining solution comprising aluminium ions, the staining strength is rather superior to when using the treatment solution comprising ammonium ions. Here, it is shown that staining time is reduced significantly.

The staining time is reduced by 10-fold (10%) when aluminium ions are used than when ammonium ions are used. It takes two hours for more than 90% of proteins to be stained.

FIG. 3 is a view illustrating the CBB-staining results of 100 μg proteins obtained from a rat brain tissue. As shown in distinct spots of FIG. 3, it is shown that proteins are well stained by the CBB-staining using the disclosed staining solution comprising aluminium ions.

When there is no protein, a stain is combined with a gel, thereby forming a background. However, in the present invention, there is no trouble in measuring a location or quantity of proteins by regulating the above-mentioned amount of aluminium ion, alcohol compound and CBB-stain solution even when destaining process is not performed.

### BRIEF DESCRIPTION OF THE DRAWINGS

FIG. 1a is a view illustrating the CBB-staining results of various proteins after a gel electrophoresis using a staining solution comprising 15% aluminium ions according to the present invention.

FIG. 1b is a view illustrating the CBB-staining results of various proteins after a gel electrophoresis using a staining solution comprising 5% aluminum ions according to the present invention.

FIG. 1c is a view illustrating the CBB-staining results of various proteins after a gel electrophoresis using a staining solution comprising ammonium ions.

FIG. 1d is a view illustrating the silver-staining results of various proteins after a gel electrophoresis.

FIG. 2 is a graph illustrating relative variation in staining strength dependent on staining time.

FIG. 3 is a view illustrating the CBB-staining results of 100 μg proteins obtained from a rat brain tissue.

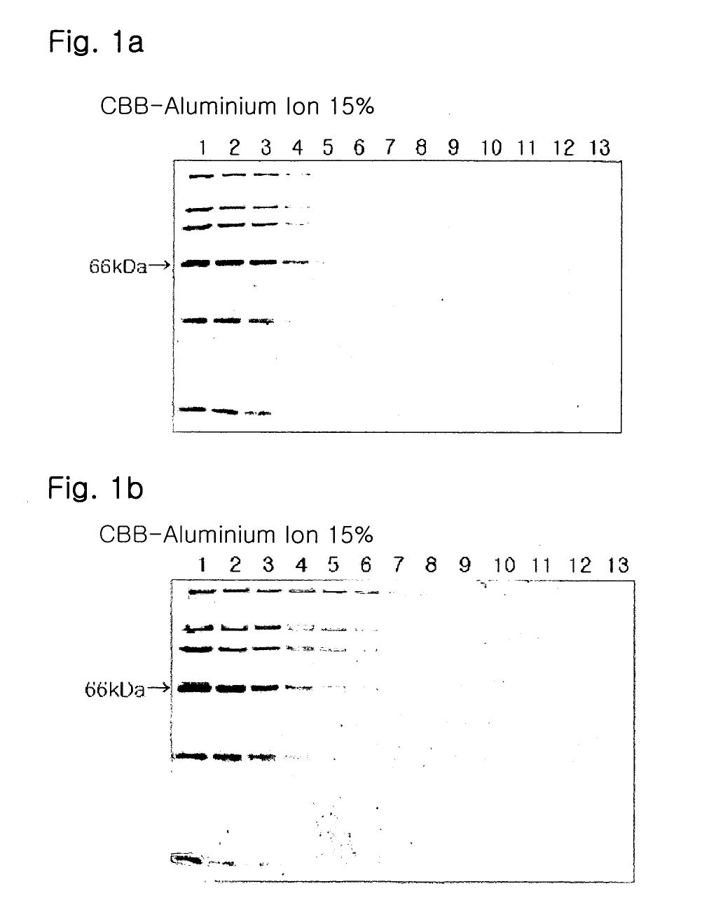

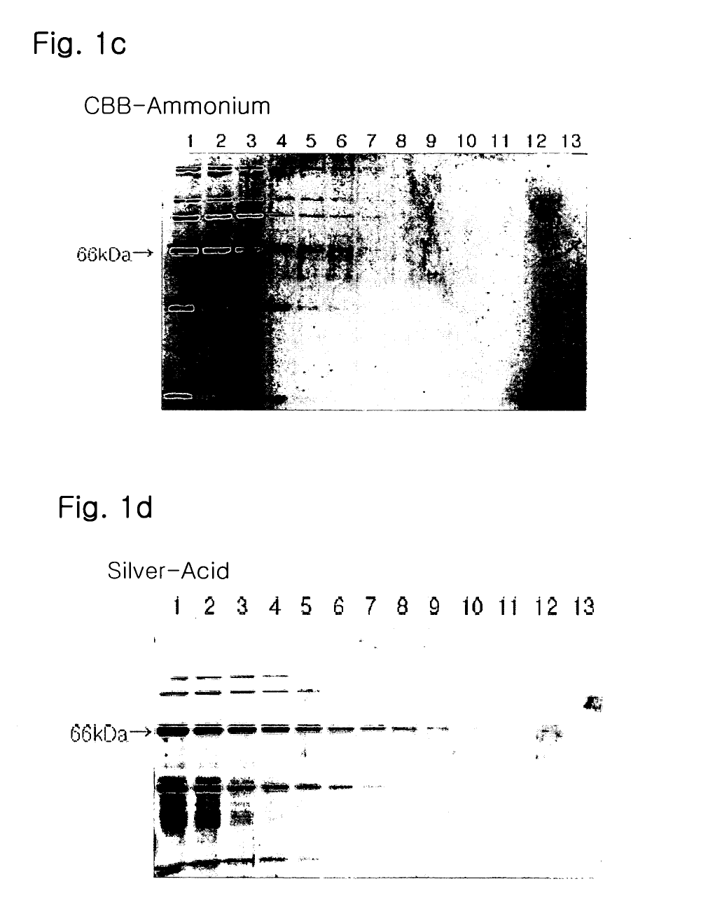

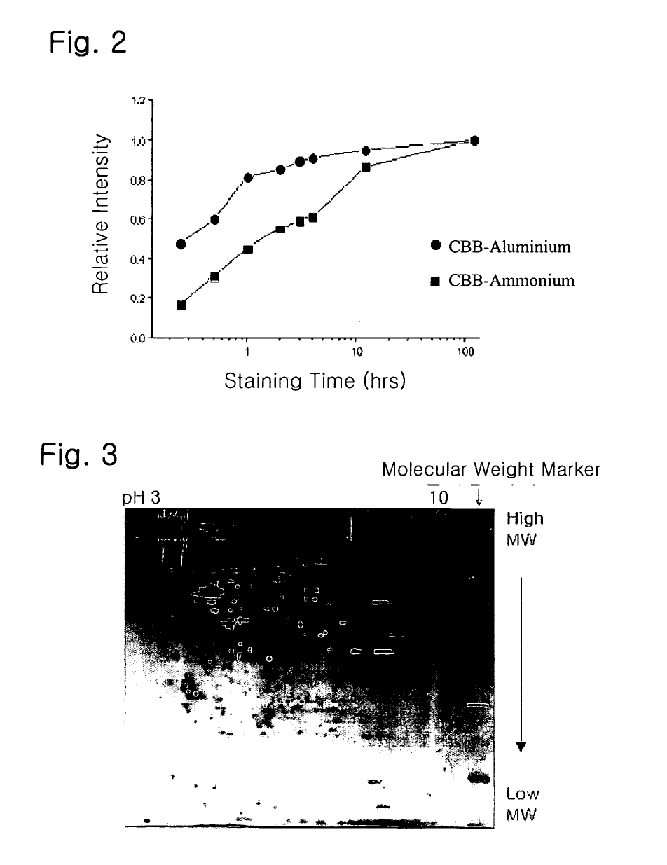

### PREFERRED EMBODIMENTS

Hereinafter, the present invention will be explained referring to a preferred embodiment. The embodiment of the present invention is shown by way of example, and not limited to the particular forms disclosed.

### EXAMPLE

After subjecting purified proteins consisting of rabbit skeletal muscle myosin, E. coli, phosphorylase b, rabbit skeletal muscle galactosidase, bovine serum albumin, egg white ovalbumin, and bovine carbonic anhydrase by electrophoresis using sodium dodecyl sulfate-polyacrylamide gel of 7×10 cm, the gel removed from a glass plate was simply washed with de-ionized water. The gel was three times treated with a primary solution comprising 30% ethanol and 2% aqueous phosphoric acid (50 ml) for 30 minutes, and three times with a secondary solution comprising 2% aqueous phosphoric acid (50 ml) for 30 minutes.

Next, the gel was soaked in a tertiary solution comprising 15% aluminium sulfate, 20% ethanol and 2% phosphate solution (50 ml) for 30 minutes. Then, a quaternary solution comprising 2% G250 and 0.2 g/L of sodium azide was added therein to have the final concentration of 1% to the whole solution (the final concentration of CBB G250 is 0.002%). Here, the disclosed CBB-staining was performed on the proteins by regulating staining time to be 120 minutes.

### Industrial Applicability

The disclosed staining solution comprising aluminum ions for CBB-staining proteins may have lower detection limit, thereby improving the sensitivity and shortening staining time and it can be performed without de-staining step.

1. A staining solution for staining proteins comprising aluminum ions, wherein the proteins are stained by a Coomassie Brilliant Blue (CBB) stain.

2. The staining solution of claim 1, wherein the aluminium ion is provided by aluminium salt selected from the group consisting of aluminium sulfate, aluminium chloride and aluminium acetate.

3. The staining solution of claim 1, wherein the aluminum ion is present in an amount ranging from 1 to 40% of the total staining solution.

4. The staining solution of claim 1, wherein the staining solution comprises an alcohol compound in an amount ranging from 5 to 40% of the total staining solution.

5. A method for staining proteins, comprising the steps of:
(a) washing a gel with deionized water, the gel obtained by performing a certain degree of purified proteins with a Sodium Dodecyl Sulfate-Polyacrylamide Gel Electrophoresis (hereinafter, referred to as “SDS-PAGE”);
(b) treating the resulting gel with a primary solution and a secondary solution;
(c) treating the resulting gel in a tertiary solution comprising aluminum ions; and
(d) adding a quaternary solution comprising a CBB stain to the resulting third solution including the gel.

6. The method of claim 5, wherein the aluminum ion is provided by aluminum salt selected from the group consisting of aluminum sulfate, aluminum chloride and aluminum acetate.

7. The method of claim 5, wherein the aluminum ion is present in an amount ranging from 1 to 40% of the third solution.

8. The method of claim 5, wherein the tertiary solution comprises an alcohol compound in an amount ranging from 5 to 40% of the tertiary solution.
